(19) United States
(12) Patent Application Publication
(10) Pub. No.: US 2019/0018195 A1
(43) Pub. Date: Jan. 17, 2019

(54) INTERCHANGEABLE FORWARD OR SIDEFIRING TIP WITH STAND OFF CATHETER

(71) Applicants: JOE D. BROWN, PANAMA CITY BEACH,, FL (US); DANIEL MALPHURS, PANAMA CITY BEACH, FL (US)

(72) Inventors: JOE D. BROWN, PANAMA CITY BEACH,, FL (US); DANIEL MALPHURS, PANAMA CITY BEACH, FL (US)

(21) Appl. No.: 16/037,784

(22) Filed: Jul. 17, 2018

Related U.S. Application Data
(60) Provisional application No. 62/533,225, filed on Jul. 17, 2017.

(51) Int. Cl. G02B 6/26; A61B 18/24

(52) U.S. Cl. CPC G02B 6/264; A61B 18/24

(57) ABSTRACT

Interchangeable tips and/or catheters are placed over a surgical laser optical fiber to allow different side and forward firing outputs. The different side firing outputs may be achieved by securing tip structures having different angled reflectors to a ferrule at the end of the fiber. The absence of any tip structures allows forward firing through the ferrule.

This application claims the benefit of Provisional U.S. Patent Appl. Ser. No. 62/533,225, filed Jul. 17, 2017, incorporated herein by reference.

### BACKGROUND OF THE INVENTION

### 1. Description of Related Art

The invention relates to interchangeable tips and/or catheters that are placed over a surgical laser optical fiber to allow different side and forward firing outputs. While tips for side firing outputs and tips for forward firing outputs are both known, it has not heretofore been proposed to provide interchangeable tips or catheters that allow different firing outputs.

By way of background, copending patent application Ser. Nos. U.S. Ser. No. 15/234,455 and U.S. 62/487,131 disclose a standoff sheath or catheter that prevents an inner side firing fiber from touching tissue, and at the time allows for rotation and longitudinal movement to provide a new lasing surface as needed throughout the procedure.

Also by way of background, copending patent application Ser. Nos. PCT/US2017/31091 and U.S. Ser. No. 13/692,512 disclose a standoff catheter that prevents a forward firing fiber from tissue touching tissue which limits the erosion of the fiber.

### DESCRIPTION OF FIGURES

FIG. 1 depicts a side firing fiber sheath 6 that slides over flat tip fiber 3. Light 1 emitting from fiber 3 is reflected by 45 degree reflector 2, which is inserted into ferrule 9 secured to the sheath 6, for example via an inner ferrule 5. Optional light channel 7 can be a hole that is small enough to let light energy through outer ferrule 9 and at the same time prevent tissue contact with reflector 2. The hole in light channel 7 can also allow the passage of air, water, D2 or other gas and fluids. The textured metal cap 4 with O-ring 8 allows for rotation of reflector 2 to a new unused area on ferrule 9. The ferrule 9 may be made of any material with appropriate optical and mechanical/thermal/structural properties, including but not limited to quartz, sapphire, PTFE, ETFE, etc.

FIG. 2 depicts an example showing interchangeable tips or catheters with three different reflectors: a 45 degree reflector 2, a convex metal cap reflector 10, and a concave metal reflector 11.

FIG. 3 depicts an example of a forward firing fiber arrangement, in which the reflector 2, or 10 or 11 has been removed from the ferrule 9, and therefore from the tip or catheter. The fiber can be advanced to be extended, flush or withdrawn (as shown in FIG. 3) relative to the sheath. Advancement allows for cleaning, whereas withdrawal prevents tissue contact.

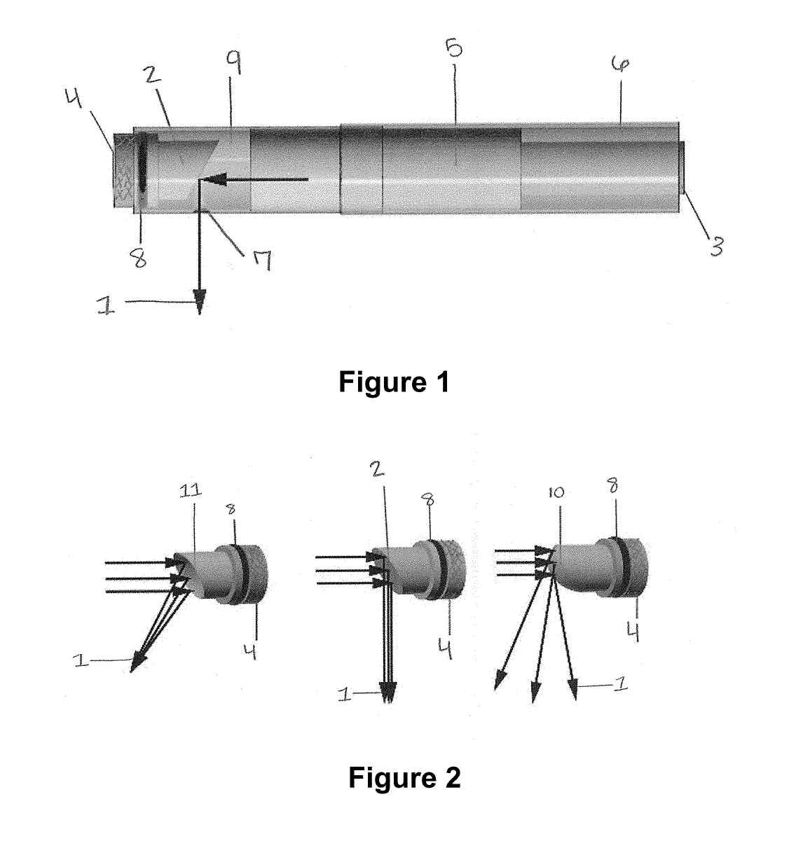

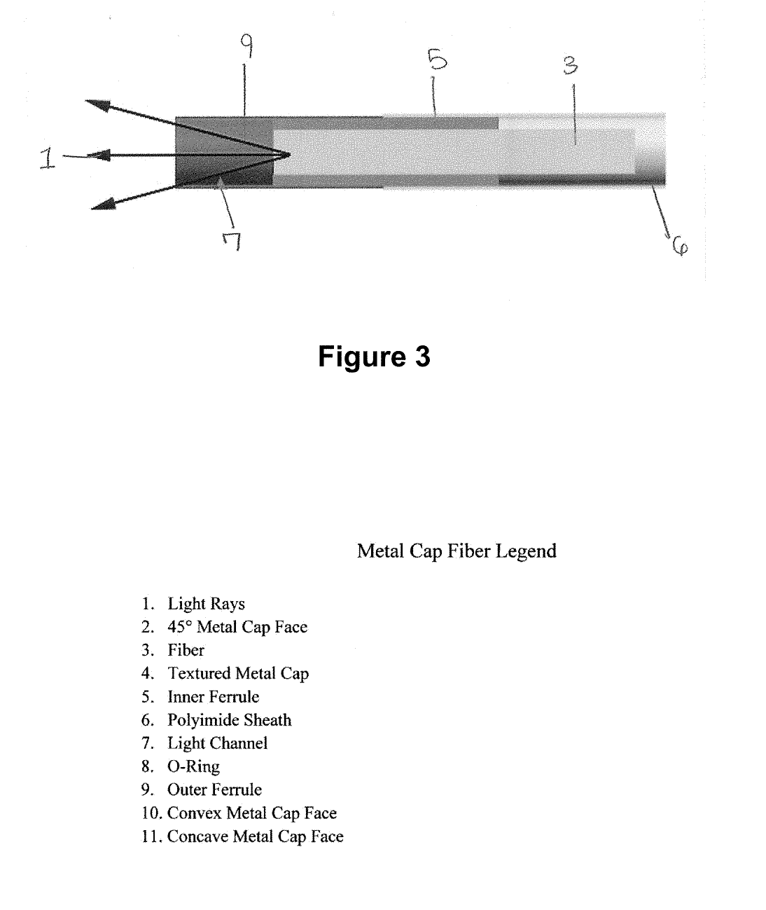

What is claimed is:

1. A protective assembly arranged to be positioned over an end of an optical fiber, comprising:
a ferrule and/or or sleeve that extends over an end of the optical fiber; and
a tip structure that adapted to be secured to the ferrule and/or sleeve, the tip structure including an angled reflective surface that deflects a laser beam emitted axially from the end of the fiber,
wherein the tip structure is interchangeable with a second tip structure having a different angled reflective surface, and
wherein removal of the tip structure results in a forward firing, non-deflected laser beam.

2. A protective assembly as claimed in claim 1, wherein the sleeve is a sheath that extends over a length of fiber, the ferrule is secured to the sheath, and the tip is inserted into an opening at the end of the ferrule.

3. A protective assembly as claimed in claim 2, wherein the ferrule is an outer ferrule secured to the sheath by an inner ferrule.

4. A protective assembly as claimed in claim 1, wherein the tip structure includes reflective surfaces of varying angle, concavity, and/or convexity.

5. A protective assembly as claimed in claim 4, wherein the reflective surfaces are metal cap faces of a metal cap.

6. A protective assembly as claimed in claim 5, wherein the metal cap is a textured metal cap with an o-ring to allow for rotation of the tip structure with respect to the ferrule, and therefore firing of the laser beam through an unused portion or area of the ferrule.

7. A protective assembly as claimed in claim 1, wherein the ferrule includes a light channel 7 that permits passage of the deflected laser beam.

8. A protective assembly as claimed in claim 7, wherein the light channel includes a hole that also allows for passage of air, water, deuterium, or other gasses and fluids.

9. A protective assembly as claimed in claim 1, wherein the fiber is movable with respect to the protective assembly, to enable advancement of the fiber for cleaning and withdrawal of the fiber for protection.

10. A protective assembly as claimed in claim 1, wherein the ferrule is made of quartz, sapphire, PTFE, or ETFE.
